(19) United States
(12) Patent Application Publication
(10) Pub. No.: US 2024/0108048 A1
(43) Pub. Date: Apr. 4, 2024

(54) PROBIOTIC AND FOLIC ACID COMPOSITIONS AND METHODS OF USING

(71) Applicant: Bonafide Health, LLC, Harrison, NY (US)

(72) Inventors: James R. Komorowski, Trumbull, CT (US); Sarah Sylla, Brooklyn, NY (US); Devon Bernsley, Harrison, NY (US)

(21) Appl. No.: 17/958,310

(22) Filed: Sep. 30, 2022

(51) Int. Cl. A23L 33/135; A61K 9/00; A61K 35/747; A61K 38/40; A61K 47/55; A61P 15/02

(52) U.S. Cl. CPC A23L 33/135; A61K 9/0053; A61K 35/747; A61K 38/40; A61K 47/551; A61P 15/02

(57) ABSTRACT

Disclosed herein are oral formulations for treating, preventing, or ameliorating symptoms associated with bacterial vaginosis or yeast infections. The oral formulations disclosed herein comprise a probiotic composition and a supplement composition formulated as a single, multi-component formulation. Also described herein are methods utilizing the aforementioned oral formulations.

### BACKGROUND

The vaginal microbiome plays an essential role in a woman's health. It is a dynamic environment and experiences variations in the composition of the microbiota during menstrual cycles and as a women ages because of changes in hormonal levels. A healthy vaginal microbiome is dominated by Lactobacillus bacteria; generally, at least 90% of the entire vaginal microbiota comprises Lactobacilli. It is believed that maintaining a healthy Lactobacilli microbiome in the vagina can aid in maintaining a healthy pH (i.e., slightly acidic, pH of about 3.8-45, as a result of the acid-producing Lactobacilli) and causing competitive crowding, thereby preventing infection and other ailments. Accordingly, a loss in abundance of these Lactobacilli can result in higher susceptibility to conditions such as yeast infections and bacterial vaginosis (BV). BV results from replacement of Lactobacilli with anaerobic organisms, such as Gardnerella vaginalis and Atopobium vaginae among others, and results in symptoms such as vaginal discharge, vaginal odor, vaginal itching, and painful urination, all of which may cause discomfort in women. BV is a common condition that affects many women globally, as BV is estimated to impact up to 30% of women in the western world, and up to 50% of women in the developing world.

Treatments for BV according to the prior art generally comprise small-molecules, such as imidazoles, and antibiotics, such as azithromycin, clarithromycin, or erythromycin. Examples of BV treatments are metronidazole, clindamycin, miconazole, tinidazole, and secnidazole. While these treatments, along with other treatments such as yeast treatments, may confer relief of BV and the symptoms associated therewith, there are high rates of recurrence of BV associated with these treatments. Recurrence rates of BV following administration of treatments of the prior art can reach as high as about 30% within 1 month and 50% within 12 months following administration of the treatment. While a definitive explanation for the high recurrence of BV associated with conventional treatments is inconclusive, one theory is that the current therapeutics indiscriminately target bacteria within the vaginal microbiome. Accordingly, the treatments of the prior art kill both the non-beneficial organisms (anaerobes, e.g., Gardnerella vaginalis and Atopobium vaginae), as well as the beneficial Lactobacilli, thus only temporarily offering relief from BV and inviting a recurrence because of a diminished Lactobacilli content within the vaginal microbiome.

To address these shortcomings, researchers have suggested the use of adjuvant Lactobacilli probiotics to offset the reduction in Lactobacilli following a primary administration of a treatment of the prior art, which have demonstrated some success in preventing BV or yeast recurrence. In other instances, researchers have suggested the use of probiotics alone, or in combination with other non-conventional treatments, i.e., those not conventionally used to treat BV or yeast, to prevent recurrence of such infections. Generally, these treatments are administered intravaginally, which may cause discomfort in a patient seeking treatment, and/or they require long durations of administration (>1 month), which may result in lower patient compliance.

In light of these discoveries, the inventors have identified a growing demand to develop formulations to treat, prevent, or ameliorate BV, yeast, or the symptoms associated therewith, while ensuring maximal patient comfort and compliance. The inventors have noted the relationship between the vaginal microbiome and its role in BV, yeast, or symptoms associated therewith. Accordingly, the inventors have found that administering an oral multi-component formulation, comprising a probiotic composition of at least one Lactobacillus strain concurrently with at least one nutritional supplement provides superior, and unexpected benefits, compared to the individual components alone. Such a finding is novel and remains to be seen in the prior art. As such, by providing a probiotic composition and a nutritional supplement together as pharmaceutical agents and/or dietary supplements, therapeutic and nutraceutical benefits can be realized, either individually, collectively, or in conjunction with other pharmaceutical agents and/or dietary supplements.

### SUMMARY

Embodiments of the present disclosure relate to novel oral formulations comprising at least one probiotic Lactobacillus bacteria and at least one nutritional supplement and their use in the amelioration, prevention, and/or treatment of the symptoms associated with BV or yeast infections.

These and other feature, aspects, and advantages of the present embodiments will become understood with reference to the following description, appended claims, and accompanying figures.

### BRIEF DESCRIPTION OF THE DRAWINGS

FIG. 1 shows a table demonstrating results from respondents that provided responses to survey questions concerning their emotions regarding the foregoing symptoms and respondents reported improvements to their levels of frustration, worry about symptoms, embarrassment, levels of affection, and levels of intimate desire.

### DETAILED DESCRIPTION

Some embodiments provide a composition comprising an amount of at least one Lactobacillus species formulated as a probiotic composition. The Lactobacillus species can be Lactobacillus jensenii, Lactobacillus rhamnosus, Lactobacillus fermentum, Lactobacillus casei, Lactobacillus salivarius, Lactobacillus gasseri, Lactobacillus plantarum, Lactobacillus acidophilus, Lactobacillus paracasei, Lactobacillus brevis, Lactobacillus reuteri, Lactobacillus bulgaricus, Lactobacillus crispatus, Lactobacillus johnsonii, Lactobacillus helveticus, or any combination thereof. Some embodiments can be formulated to have varying amounts of these one or more Lactobacillus species.

In some embodiments, a probiotic composition can comprise a first Lactobacillus species and a second Lactobacillus species, wherein the first Lactobacillus species and the second Lactobacillus species are formulated in a ratio. In certain embodiments, a ratio of the first Lactobacillus species and the second Lactobacillus species can be about 4:1 In certain embodiments, a ratio of the first Lactobacillus species and the second Lactobacillus species can be about 1:1. In some embodiments, a ratio of the first Lactobacillus species and the second Lactobacillus species can be within the range of about 10:1 to about 1:10. In this regard, a ratio of the first Lactobacillus species and the second Lactobacillus species may be about 2:1, about 3:1, about 4:1, about 5:1, about 6:1, about 7:1, about 8:1, about 9:1, about 10:1, about 1:2, about 1:3, about 1:4, about 1:5, about 1:6, about 1:7, about 1:8, about 1:9, or about 1:10.

In some embodiments, a probiotic composition can comprise an amount of at least one Lactobacillus species of about 1 billion CFU to about 20 billion CFU, or any ratio in between. For example, in some embodiments the amount of the at least one Lactobacillus species can be about 1 billion CFU to about 4 billion CFU. Some embodiments can comprise an amount of at least one Lactobacillus species of about 4 billion or more CFU. In certain embodiments, the amount of the at least one Lactobacillus species can be about 4 billion CFU to about 10 billion CFU. In some embodiments, the amount of the at least one Lactobacillus species can be about 10 billion CFU to about 20 billion CFU.

In some embodiments, a probiotic composition can comprise an amount of a first Lactobacillus species and a second Lactobacillus species. In certain embodiments, the amount of both the first Lactobacillus species and second Lactobacillus species can be about 1 billion CFU to about 5 billion CFU. Some embodiments can comprise an amount of a first Lactobacillus species and a second Lactobacillus species of about 5 billion or more CFU. In certain embodiments, the amount of both the first Lactobacillus species and second Lactobacillus species can be about 5 billion CFU to about 10 billion CFU. In some embodiments, the amount of both the first Lactobacillus species and second Lactobacillus species can be about 10 billion CFU to about 20 billion CFU. In certain embodiments, the amount of the first Lactobacillus species can be about 1 billion CFU to about 5 billion CFU and second Lactobacillus species can be about 5 billion CFU to about 10 billion CFU. In some embodiments, the amount of the first Lactobacillus species can be about 1 billion CFU to about 5 billion CFU and second Lactobacillus species can be about 10 billion CFU to about 20 billion CFU. In some embodiments, the amount of the first Lactobacillus species can be about 5 billion CFU to about 10 billion CFU and second Lactobacillus species can be about 1 billion CFU to about 5 billion CFU. In certain embodiments, the amount of the first Lactobacillus species can be about 5 billion CFU to about 10 billion CFU and second Lactobacillus species can be about 10 billion CFU to about 20 billion CFU. In some embodiments, the amount of the first Lactobacillus species can be about 10 billion CFU to about 20 billion CFU and second Lactobacillus species can be about 1 billion CFU to about 5 billion CFU. In certain embodiments, the amount of the first Lactobacillus species can be about 10 billion CFU to about 20 billion CFU and second Lactobacillus species can be about 5 billion CFU to about 10 billion CFU.

In some embodiments, an oral formulation, as described herein, may comprise at least one probiotic Lactobacillus bacteria and at least one nutritional supplement, as described herein. Certain embodiments comprise a method of balancing the vaginal microbiome, the method comprising: identifying a subject with a pH level above about 4.5; administering an oral multi-component formulation, wherein the oral multi-component formulation comprises: a probiotic composition layer, wherein the probiotic composition layer comprises an amount of at least one Lactobacillus species; a supplement composition layer, wherein the supplement composition layer comprises an amount of at least one nutritional supplement ingredient; and a middle barrier layer disposed between the probiotic composition layer and the supplement composition layer, wherein the probiotic composition layer and the supplement composition layer are positioned on opposing sides of the middle barrier layer.

Certain embodiments comprise a method of regulating, restoring, or balancing the vaginal pH of a subject, the method comprising: identifying a subject in need thereof; administering an oral formulation of one or more formulations as set forth herein. In some embodiments, the oral formulation is a multi-component formulation. In some embodiments, the oral formulation comprises one or more of a probiotic composition and a nutritional supplement.

In some embodiments, a probiotic composition may further comprise an amount of at least one additional additive that reduces sensitivity to moisture and/or prolongs shelf-life of the probiotic composition. In some embodiments, the amount of the at least one additional additive can be between about 10 mg and about 500 mg. For example, the amount of the at least one additional additive in the probiotic composition may be about 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, or any range or amount in between any two of the preceding values and any other ranges or amounts disclosed herein. In certain embodiments, the at least one additional additive may be lactoferrin. In certain embodiments, the additional additive may be lactoferrin, and the probiotic composition may comprise an amount of lactoferrin between about 10 mg and about 500 mg. For example, the amount of lactoferrin in the probiotic composition can be about 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, or any range or amount in between any two of the preceding values and any other ranges or amounts disclosed herein.

Some embodiments provide a composition comprising at least one nutritional supplement ingredient formulated as a supplement composition.

In certain embodiments, the at least one nutritional supplement ingredient can be an essential fatty acid, such as linolenic acid or linoleic acid, or an essential amino acid, such as tryptophan, lysine, methionine, phenylalanine, threonine, valine, leucine, isoleucine, arginine, or histidine, or any combination thereof.

In some embodiments, the at least one nutritional supplement ingredient can be a vitamin, such as retinol (vitamin A), thiamine (vitamin B1), riboflavin (vitamin B2), niacin (vitamin B3), pantothenic acid (vitamin B5), pyridoxine, pyridoxamine, or pyridoxal (vitamin B6), biotin (vitamin B7) or pharmaceutically acceptable salts thereof, folic acid (vitamin B9) or pharmaceutically acceptable salts thereof, cobalamin (vitamin B12), choline, ascorbic acid (vitamin C) or pharmaceutically acceptable salts thereof, ergocalciferol (vitamin D2), calciferol (vitamin D3), 22-dihydroergocalciferol (vitamin D4), sitocalciferol (vitamin D5), tocopherol (vitamin E), phylloquinone (vitamin K1), menaquinone (vitamin K2), menadione (vitamin K3), or any combination of the foregoing.

In some embodiments, the at least one nutritional supplement ingredient can be a dietary mineral, such as chromium, including, chromium polynicotinate, chromium acetate, chromium histidinate, chromium nicotinate, chromium chloride, and the like, or any or any pharmaceutically acceptable salts, hydrates, solvates, or mixtures thereof, bromine, cobalt, copper, fluorine, germanium, iodine, iron, magnesium, manganese, molybdenum, potassium, selenium, silicon, zinc, calcium, phosphorous, sodium, sulfur, vanadium, or any combination thereof.

In certain embodiments, the at least one nutritional supplement ingredient can be n-acetyl cysteine, nicotinamide riboside, resveratrol, NAD+ precursors, Coenzyme Q10, reduced Coenzyme Q10, omega-3-fatty acids, bee pollen, cabbage powder, pterostilbene, nicotinamide mononucleotide, cranberry extract, turmeric or an extract thereof, royal jelly, açai berry, beet root or an extract thereof, coral calcium, oyster shell, Gotu kola or an extract thereof, Gingko biloba or an extract thereof, lions mane mushroom, pomegranate, hibiscus flower, strawberry powder, dandelion root, celery powder, parsley powder, peppermint leaf, cinnamon bark powder, maca root, phycocyanin or an extract thereof, pumpkin seed or an extract thereof, Crataeva mirvala or an extract thereof, Lindera aggregata or an extract thereof, citrulline nitrate, creatine nitrate, beta-alanine, beta-alanine nitrate, or any combination thereof.

In some embodiments, the amount of the nutritional supplemental ingredient, disclosed herein, can be about 10 μg to about 1 g. For example, the amount of the composition can be about 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 125 μg, 150 μg, 175 μg, 200 μg, 225 μg, 250 μg, 275 μg, 300 μg, 325 μg, 350 μg, 375 μg, 400 μg, 425 μg, 450 μg, 475 μg, 500 μg, 525 μg, 575 μg, 600 μg, 625 μg, 650 μg, 675 μg, 700 μg, 725 μg, 750 μg, 775 μg, 800 μg, 825 μg, 850 μg, 875 μg, 900 μg, 925 μg, 950 μg, 975 μg, 1000 μg, 5 mg, 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, or any range or amount in between any two of the preceding values and any other ranges or amounts disclosed herein.

Some embodiments can be formulated as a multi-component formulation that can comprise a nutritional supplement and/or a probiotic composition, as disclosed herein, disposed as the center layer of the formulation, a nutritional supplement composition, as disclosed herein, disposed as the outer layer of the formulation, and a middle barrier layer disposed therebetween, wherein the probiotic composition layer and the nutritional supplement composition layer are positioned on opposing sides of the middle barrier layer. A multi-component formulation can be formulated as a tablet, a hard capsule, a soft capsule, a plant-derived capsule, gel capsule, caplet, or the like. The probiotic composition can be formulated as a solid, such as a powder, granule, amorphous solid, crystalline solid, or the like, and the nutritional supplement can be formulated as a solid, such as a powder, granule, amorphous solid, crystalline solid or the composition can be formulated as a liquid, such as an aqueous solution, an oil-based solution, an aqueous suspension, an oil suspension, an emulsion, a syrup, an elixir, or the like. Some embodiments can comprise a combination of the foregoing forms of formulations.

In certain embodiments, a multi-component formulation can be formulated as a tablet, a hard capsule, a soft capsule, a plant-derived capsule, gel capsule, caplet, or the like. The multi-component formulation can comprise a probiotic composition disposed as a layer, a nutritional supplement composition disposed as a layer, and a barrier layer disposed therebetween, wherein the probiotic composition layer and the supplement composition layer are positioned on opposing sides of the middle barrier layer. The probiotic composition can be formulated as a solid, such as a powder, a granule, particles, an amorphous solid, a crystalline solid, or the like, and the nutritional supplement can be formulated as a solid, such as a powder, granule, amorphous solid, crystalline solid or the composition can be formulated as a liquid, such as an aqueous solution, an oil-based solution, an aqueous suspension, an oil suspension, an emulsion, a syrup, an elixir, or the like. Some embodiments can comprise a combination of the foregoing forms of formulations.

In certain embodiments, the middle barrier layer, as described herein, can be comprised of excipients that would separate the probiotic composition from the supplement composition. The barrier layer can be included in the formulation by art-recognized coating or layering procedures. The barrier layer may be comprised of pharmaceutically acceptable compounds such as, for example, sugar, polyethylene glycol, polyvinylpyrrolidone, polyvinyl alcohol, polyvinyl acetate, hydroxypropyl cellulose, methylcellulose, ethylcellulose, hydroxypropyl methylcellulose, carboxymethylcellulose sodium and others, used alone or in mixtures. Additives such as plasticizers, colorants, pigments, fillers, anti-tacking and anti-static agents including magnesium stearate, titanium dioxide, talc, and other additives can optionally be included in the barrier layer. In some embodiments, the middle barrier portion is comprised of a controlled release, delayed release, or enteric coating to control or delay the release of the inner contents of the formulation.

In some embodiments, the multi-component formulation, as described herein, includes an outer coating. The outer coating serves to provide an immediate release, controlled-release, delayed-release, or enteric-coating to control or delay the release of the supplement composition and probiotic composition. The outer coating layer (or layers) can be applied by coating or layering procedures which are well-established in the relevant arts. The outer coating layer can be at least one of a pharmaceutically acceptable compound selected from the group consisting of sugar, polyethylene glycol, polyvinylpyrrolidone, polyvinyl alcohol, polyvinyl acetate, hydroxypropyl cellulose, methylcellulose, ethylcellulose, hydroxypropyl methylcellulose, carboxymethylcellulose sodium and combinations thereof. Additives including plasticizers, colorants, pigments, fillers, anti-tacking, and anti-static agents can optionally be included in the outer coating. In some embodiments, the outer coating regulates release of the supplement composition and/or probiotic composition from the tablet, capsule, caplet, or the like. Controlled-release, delayed-release, and/or enteric-coating technology is well-established in the pharmaceutical and formulation arts. Compositions described herein can be formulated as sustained or slow release, designed to be released into the gastrointestinal tract of a patient at a specified rate of delivery.

In some embodiments, a probiotic composition, a supplement composition, a middle barrier layer, and/or an outer coating, as described herein, may include one or more excipients, such as the following classes of agents: sweeteners, flavoring agents, coloring agents, coatings, preservatives, lubricants, binders, surfactants, fillers, disintegrants, suspending agents, dispersing agents, wetting agents, thickening agents. In certain embodiments, the one or more excipients may be one or more of glycerin, hydroxypropyl methylcellulose, microcrystalline cellulose, magnesium stearate, silicon dioxide, calcium carbonate, sodium carbonate, lactose, calcium phosphate, sodium phosphate, corn starch, alginic acid, starch, gelatin, acacia, stearic acid, talc, glyceryl monostearate, glyceryl distearate, kaolin, peanut oil, liquid paraffin, olive oil, arachis oil, sesame oil, coconut oil, sucrose, saccharin, beeswax, hard paraffin, xanthan gum, cetyl alcohol, sorbitol, or any combination of the foregoing. The excipients that may be included in any composition, a middle layer, or a coating, as described herein, are not particularly limited.

In certain embodiments, a multi-component formulation comprising a probiotic composition and a supplement composition, according to the disclosure, can be administered to a subject to treat, ameliorate, or prevent BV or yeast infections. In some embodiments, a multi-component formulation comprising a probiotic composition and a supplement composition, as described herein, can be administered to a subject to treat, ameliorate, or prevent in a subject experiencing symptoms associated with BV or yeast infections, such as vaginal odor, vaginal itching, vaginal discharge, painful urination, or any combination thereof, even if the subject does not have BV or a yeast infection.

In some embodiments, a multi-component formulation comprising a probiotic composition and a supplement composition, according to the disclosure, can be administered to a subject to restore or maintain healthy or optimal levels of Lactobacilli within the vaginal microbiome.

In some embodiments, a multi-component formulation comprising a probiotic composition and a supplement composition to prevent BV/yeast, vaginal odor, vaginal itching, vaginal discharge, painful urination, or any combination thereof, as described herein, can be administered to a subject at least once daily for a periodic cycle. In certain embodiments, a periodic cycle of at least once daily administration can comprise administering the formulation to a subject once a day for about 2 consecutive days to about 20 consecutive days. For example, a periodic cycle of at least once daily administration can comprise administering the formulation to a subject once a day for about 15 consecutive days. In some embodiments, the periodic cycle of at least once daily administration may be repeated on a monthly basis. For example, a periodic cycle of at least once daily administration may comprise administering the formulation to a subject once a day for about 15 consecutive days, and the periodic cycle may be repeated monthly for at least one month consecutively, six months consecutively, twelve months consecutively, or longer. A dosage cycle can be one dose per month, one dose every 15 days, one dose every 10 days, one dose every 5 days, one dose ever two days, or daily dosage.

In certain embodiments, treating, ameliorating, or preventing yeast/BV, vaginal odor, vaginal itching, vaginal discharge, painful urination, or any combination thereof, may result in symptomatic improvement in a subject after about one periodic cycle, as described herein. For example, after being administered a formulation, as disclosed herein, for 15 consecutive days for 1 month consecutively, a subject may observe amelioration and/or treatment of BV or symptoms associated therewith. Additionally, after being administered a formulation, as disclosed herein, for 15 consecutive days for 1 month consecutively, a subject may be more likely to be prevented from contracting BV or experiencing a symptom associated therewith.

Without being bound by any particular theory, it is believed that yeast infections, BV or the symptoms associated therewith result from a reduction in the concentration of Lactobacilli within the vaginal microbiota. Accordingly, it is believed that by supplementing the vaginal microbiota with Lactobacilli, BV, yeast or its associated symptoms can be treated, prevented, ameliorated. It is believed that the embodiments disclosed herein maintain and/or restore concentration of Lactobacilli within the vaginal microbiome to healthy or optimum levels through multiple mechanisms.

First, it is believed that providing at least one Lactobacillus strain effectively restores the Lactobacilli content of the vaginal microbiome by directly increasing the Lactobacilli content, and in some instances, by controlling growth of other microorganisms (non-Lactobacilli) within the vaginal flora. For example, in some embodiments, the at least one Lactobacillus strain may be Lactobacillus acidophilus, which ferments sugar to produce lactic acid and thrives in an acidic environment (pH<5). Both the lactic acid compound and a low pH (a consequence of circulating lactic acid) may be hostile to non-Lactobacilli microorganisms, thereby increasing the relative concentration of Lactobacilli in the vaginal microbiome.

Second, by co-administering at least one Lactobacillus strain with a nutritional supplement, it is believed that that the nutritional supplement increases the effective amount of Lactobacilli delivered to the vagina. It is also believed that the nutritional supplement increases the viability of the at least one Lactobacillus strain once in the body of a subject. For example, in some embodiments, the nutritional supplement may be folic acid or a pharmaceutically acceptable salt thereof. It is believed that folic acid or a pharmaceutically acceptable salt thereof increases the amount (CFU count) of the Lactobacillus strain(s) once the formulation is within the body and the nutritional supplement and Lactobacillus strain(s) “mix” (they are separated in different compartments within the multi-component formulation), such that the amount (CFU count) of the Lactobacillus strain(s) is significantly higher in the presence of folic acid or a pharmaceutically acceptable salt thereof compared to the an amount (CFU count) of the Lactobacillus strain(s) that would exist were no folic acid or a pharmaceutically acceptable salt thereof present. This increase results in a higher “effective dose” in the formulation, and therefore a higher dose being delivered to the vagina of the individual receiving treatment. It is also believed that the nutritional supplement may decrease the barriers for delivery of the at least one Lactobacillus strain. For example, in some embodiments, the nutritional supplement may be folic acid or a pharmaceutically acceptable salt thereof. It is believed that folic acid or a pharmaceutically acceptable salt thereof may increase the permeability of the gastrointestinal tract. Accordingly, a higher permeability results in a larger uptake of the Lactobacillus strain(s) at the desired site (i.e., the vagina), thereby increasing the “effective dose”.

Third, by incorporating additional additives that reduce sensitivity to moisture with the at least one Lactobacillus strain, it is believed that the effective amount of Lactobacilli in a formulation remains at a higher level than what would were no additional additive present. For example, in some embodiments, the probiotic composition may further comprise lactoferrin. It is believed that lactoferrin increases the stability of the Lactobacillus strain(s), such that the viability of the Lactobacillus strain(s), and therefore amount (CFU count), is higher when stored in the presence of lactoferrin, compared to Lactobacillus strain(s) stored absent lactoferrin. Accordingly, this increase results in a higher “effective dose” in the formulation, and therefore a higher dose being delivered to the vagina of the individual receiving treatment

As used herein, the term “barrier layer” refers to an impermeable structural element of the multi-component formulation that segregates the probiotic composition and the supplement composition from each other to the extent that the two compositions do not mix.

As used herein, the term “identifying” refers to detecting or selecting a subject from a population of potential subjects, for example, to establish that a particular subject possesses certain properties or characteristics. “Identifying” may include, for example, self-identification, self-diagnosis, and diagnosis by a medical professional.

As used herein, the terms “preventing,” “treating,” “treatment,” “ameliorating,” and the like are used herein to generally refer to obtaining a desired pharmacological and physiological effect and can also refer to a nutritional or nutraceutical effect, the scopes and meanings of which will be clear to the skilled artisan based upon the context in which these terms are used. The effect may be prophylactic in terms of preventing or partially preventing a disease, symptom, or condition thereof and/or may be therapeutic in terms of a partial or complete cure of a disease, condition, symptom, or adverse effect attributed to the disease. The term “treatment” as used herein encompasses any treatment of a disease in a mammal, particularly a human and includes: (a) preventing the disease from occurring in a subject which may be predisposed to the disease but has not yet been diagnosed as having it; (b) inhibiting the disease or arresting its development; or (c) relieving the disease, causing regression of the disease and/or its symptoms, conditions, and co-morbidities.

As used herein, the meaning of the terms “vaginal odor,” “vaginal itching,” “vaginal discharge,” and “painful urination” would immediately be envisaged by the skilled artisan. The etiology of these terms would be understood by the skilled artisan to refer to the common symptoms resulting from BV or yeast. However, compositions described herein can also be administered to treat, ameliorate, prevent, or reduce vaginal odor, vaginal itching, vaginal discharge, and painful urination that are not caused by BV or yeast infections.

As used herein, the term “excipient” refers to any compound that is part of a formulation that is not an active ingredient, i.e., one that has no relevant biological activity, and which is added to the formulation to provide specific characteristics to the dosage form, including by way of example, providing protection to the active ingredient from chemical degradation, facilitating release of a tablet or caplet from equipment in which it is formed, and so forth.

As used herein, the term “restoring” or “maintaining” refers to using the formulations, as described herein, to enact a physiological change within a subject or patient. In some embodiments, “restoring” or “maintaining,” “healthy” or “optimal” levels of Lactobacilli within the vagina microbiome refers to using the formulations, as described herein, to enact a physiological change within a subject or patient, wherein the physiological change results in the subject or patient developing a vaginal microbiome that comprises at least 90% Lactobacilli. “Restoring,” “maintaining,” “healthy” or “optimal” levels of Lactobacilli within the vagina microbiome can also refer to using the formulations, as described herein, to cause a vaginal pH to remain or return to a particular pH, i.e., between about 3.8 and 5.0. In some embodiments, “restoring” or “maintaining,” “healthy” or “optimal” levels of Lactobacilli within the vagina microbiome refers to using the formulations, as described herein, to enact a physiological change within a subject or patient, wherein the physiological change results in the vaginal microbiome of the subject or patient having a substantially similar vaginal microbiome composition compared to the subject's or patient's vaginal microbiome prior to the subject or patient experiencing yeast, BV, symptoms associated therewith, or both.

To provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

In addition, the appropriate dosage of the compositions can depend, for example, on the condition to be treated, the severity and course of the condition, whether the composition is administered for preventive or therapeutic purposes, previous therapy, the patient's clinical history and response to the composition, the type of composition used, and the discretion of the attending physician. The composition can be suitably administered to the patient at one time or over a series of treatments and may be administered to the patient at any time from diagnosis onwards. The composition may be administered as the sole treatment or in conjunction with other drugs or therapies useful in treating the condition in question.

The present disclosure comprises supplement compositions and probiotic compositions useful for the treatment, prevention, and/or amelioration of BV/yeast and the systems associated therewith. Some embodiments provide a multi-component formulation comprising a supplement composition and a probiotic composition, as disclosed herein. Embodiments comprising a probiotic composition and supplement composition, described herein, present said compositions in an unnatural form, i.e., the probiotic composition and supplement composition comprise components that are present in a form that is different than what occurs naturally.

For the purpose of this disclose, “subject” or “patient”, as used herein, refers to a human female. In certain embodiments, the “subject” or “patient” may have BV or yeast, whether or not they show symptoms associated with BV or yeast. In other embodiments, the “subject” or “patient” may be experiencing symptoms such as vaginal odor, vaginal itching, vaginal discharge, and/or painful urination, whether or not they are diagnosed with an infection. In some embodiments, the “subject” or “patient” may not have an infection, nor associated symptoms, but may have had BV, yeast, or associated symptoms previously. In all instances, embodiments disclosed herein are effective in treating or ameliorating the symptoms of the “subject” or “patient”, BV, yeast, or both, and are effective in preventing future symptoms of the “subject” or “patient”, BV, yeast or both.

The present disclosure comprises nutritional and therapeutic oral formulations comprising a probiotic composition and a supplement composition, and methods of using the same. Some embodiments provide solid dosage forms of the compositions disclosed herein. Some embodiments provide aqueous solutions of compositions disclosed herein. Embodiments described herein comprising compositions disclosed herein means that the composition disclosed herein is present in an unnatural form that is different from that which occurs naturally (e.g., in a pill, capsule, and the like formulated as layers present as a powder, solution, and the like), or the oral formulation, probiotic composition, or supplement composition results in unnatural supplementation that is unachievable through a non-supplemented diet.

### EXAMPLES

### Example 1

An open-label experience trial was be performed to determine the efficacy of treatments for BV or symptoms associated therewith. The experience trial participants received a treatment for BV. Participants took an oral formulation, which comprised 400 mcg Folate (L-5-Methyltetrahydrofolate), 5 billion CFU of a Lactobacilli blend (e.g., 4 billion CFU Lactobacillis acidophilus and 1 billion CFU Lactobacillus rhamnosus), and 50 mg of Lactoferrin, which was administered once daily and the results were measured as set forth herein.

The experience trial started with 31 women and evaluated safety and efficacy endpoints with a vulvovaginal symptoms questionnaire (VSQ) that was sent out at baseline and every two weeks, over the course of 3-6 months. The VSQ collected a rating from trial participants on the severity of vaginal symptoms including vaginal itching, discharge, burning, and odor using a four level scale (absent; mild; moderate; severe). The demographics of the subjects was as follows:
Age: 47.3+/−15.1 yrs
Height: 63.4+/−2.2 inches
Body Weight: 159.3+/−28.8 lbs
BMI (kg/m2): 28.0+/−5.4
22 White/Caucasian, 3 Black/African American, 6 Hispanic

Regarding efficacy, the clinical data demonstrated that the treatment showed that they are are efficacious at reducing the symptoms listed on the VSQ. The clinical data also demonstrated that the evaluated treatment was safe. Herein, “safe” refers to the treatment having a statistically insignificant prevalence of symptoms and/or adverse events from the treatment.

The results obtained in the treatment were as follows:

When asked if “during the past week have you experienced odor from your vulva or vagina,” respondents (n=31) reported significant reductions at weeks 2, 4, 6, 8, 10, and 12 (p<0.001) from baseline (Week 0), as follows:

| | Timepoint | | | | | | |
|---|---|---|---|---|---|---|---|
| | W 0 | W 2 | W 4 | W 6 | W 8 | W 10 | W 12 |
| Reporting odor (yes) | 31/31 | 14/31 | 16/31 | 10/31 | 12/31 | 12/31 | 8/31 |
| % Change from | | −54.8% | −48.4% | −67.7% | −61.3% | −61.3% | −74.2% |
| Baseline | | | | | | | |

When asked to rate the severity of their vaginal odor over the past week (absent, mild, moderate, severe), respondents (n=31) starting with a baseline reporting of mild to severe symptoms reported significant reductions at weeks 2, 4, 6, 8, 10, and 12 (p<0.001) as follows:

| Timepoint | | | | | | |
|---|---|---|---|---|---|---|
| W 0 | W 2 | W 4 | W 6 | W 8 | W 10 | W 12 |

| | | | | | | |
|---|---|---|---|---|---|---|
| Decrease in severity | 67% | 77% | 86% | 77% | 83% | 87% |
| from baseline | (n = 30) | (n = 30) | (n = 29) | (n = 30) | (n = 29) | (n = 31) |

### Mild, Moderate, and Severe Subjects Reporting a Reduction.

When asked to rate the severity of their vaginal odor over the past week (absent, mild, moderate, severe), respondents (n=21) starting with a baseline reporting of moderate to severe symptoms reported significant reductions at weeks 2, 4, 6, 8, 10, and 12 (p<0.001) as follows:

| Timepoint | | | | | | |
|---|---|---|---|---|---|---|
| W 0 | W 2 | W 4 | W 6 | W 8 | W 10 | W 12 |

| | | | | | | |
|---|---|---|---|---|---|---|
| Decrease in severity | 70% | 86% | 95% | 90% | 95% | 95% |
| from baseline | (n = 20) | (n = 21) | (n = 20) | (n = 20) | (n = 19) | (n = 21) |

### Moderate and Severe Subjects Reporting a Reduction

When asked to rate the severity of their vaginal itching over the past week (absent, mild, moderate, severe), respondents (n=15) starting with a baseline reporting of mild to severe symptoms reported significant reductions at weeks 6, 8, 10, and 12 (p<0.01) as follows:

| Timepoint | | | | | | |
|---|---|---|---|---|---|---|
| W 0 | W 2 | W 4 | W 6 | W 8 | W 10 | W 12 |

| | | | | | | |
|---|---|---|---|---|---|---|
| Decrease in severity | 53% | 71% | 85% | 87% | 87% | 93% |
| from baseline | (n = 15) | (n = 14) | (n = 13) | (n = 15) | (n = 15) | (n = 15) |

### Mild, Moderate, and Severe Subjects Reporting a Reduction

When asked to rate the severity of their vaginal irritation over the past week (absent, mild, moderate, severe), respondents (n=19) starting with a baseline reporting of mild to severe symptoms reported significant reductions at weeks 10 and 12 (p<0.01) as follows:

| Timepoint | | | | | | |
|---|---|---|---|---|---|---|
| W 0 | W 2 | W 4 | W 6 | W 8 | W 10 | W 12 |

| | | | | | | |
|---|---|---|---|---|---|---|
| Decrease in severity | 72% | 84% | 83% | 89% | 94% | 95% |
| from baseline | (n = 18) | (n = 19) | (n = 18) | (n = 19) | (n = 17) | (n = 19) |

### Mild, Moderate, and Severe Subjects Reporting a Reduction

When asked to rate the severity of their vaginal dryness over the past week (absent, mild, moderate, severe), respondents (n=25) starting with a baseline reporting of mild to severe symptoms reported significant reductions at weeks 4 and 12, along with a sustained reduction in the severity of the dryness over weeks two through 12, as follows:

| Timepoint | | | | | | |
|---|---|---|---|---|---|---|
| W 0 | W 2 | W 4 | W 6 | W 8 | W 10 | W 12 |

| | | | | | | |
|---|---|---|---|---|---|---|
| Decrease in severity | 54% | 72% | 68% | 68% | 65% | 72% |
| from baseline | (n = 24) | (n = 25) | (n = 25) | (n = 25) | (n = 23) | (n = 25) |

### Mild, Moderate, and Severe Subjects Reporting a Reduction

When asked to rate the severity of their vaginal burning over the past week (absent, mild, moderate, severe), respondents (n=14) starting with a baseline reporting of mild to severe symptoms reported significant reductions at weeks 4, 8, 10 and 12 (p<0.05) as follows:

| Timepoint | | | | | | |
|---|---|---|---|---|---|---|
| W 0 | W 2 | W 4 | W 6 | W 8 | W 10 | W 12 |

| | | | | | | |
|---|---|---|---|---|---|---|
| Decrease in severity | 77% | 79% | 71% | 86% | 83% | 79% |
| from baseline | (n = 13) | (n = 14) | (n = 14) | (n = 14) | (n = 12) | (n = 14) |

### Mild, Moderate, and Severe Subjects Reporting a Reduction

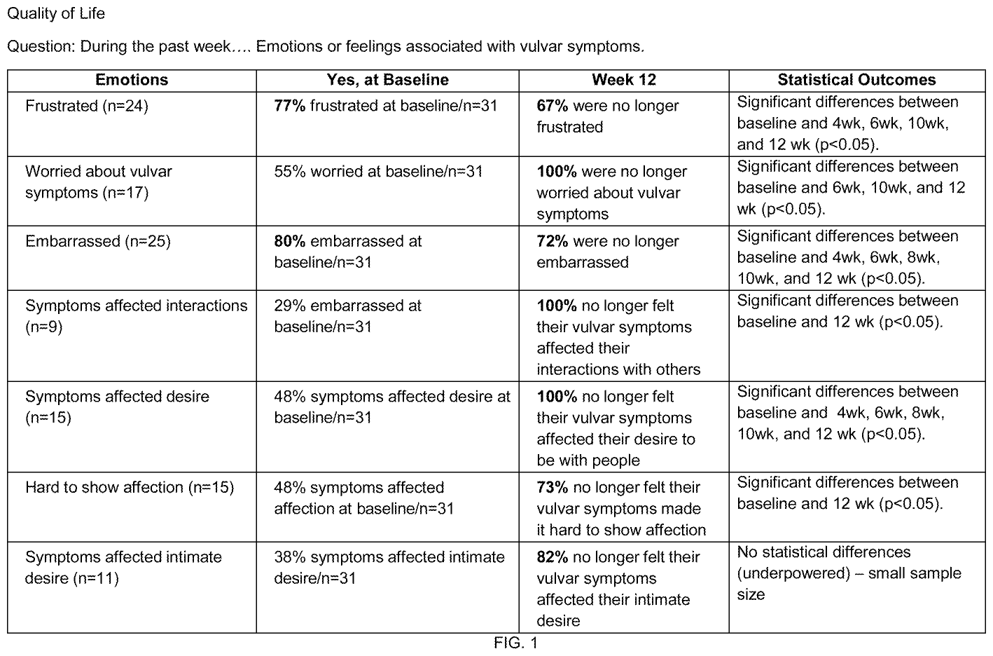

Respondents also provided responses to survey questions concerning their emotions regarding the foregoing symptoms and respondents reported improvements to their levels of frustration, worry about symptoms, embarrassment, levels of affection, and levels of intimate desire. These results are shown in FIG. 1.

Additional testing will be conducted following similar protocols wherein compounds of the disclosure will be administered to subjects to ascertain improvements in the severity of vaginal symptoms including itching, discharge, burning, odor, pain during sexual intercourse, spotting (bleeding) during sexual intercourse, along with other metrics such as sexual desire and pleasure during intercourse.

While the present invention has been described in some detail for purposes of clarity and understanding, one will appreciate that the present invention has been described in some detail for purposes of clarity and understanding, one will appreciate that various changes in form and detail can be made without departing from the true scope of the invention.

1. An oral multi-component formulation, the multi-component formulation comprising:
a probiotic composition layer, wherein the probiotic composition layer comprises an amount of at least one Lactobacillus species;
a supplement composition layer, wherein the supplement composition layer comprises an amount of at least one nutritional supplement ingredient; and
a middle barrier layer disposed between the probiotic composition layer and the supplement composition layer, wherein the probiotic composition layer and the supplement composition layer are positioned on opposing sides of the middle barrier layer.

2. The oral multi-component formulation of claim 1, wherein the probiotic composition layer comprises an amount of Lactobacillus acidophilus and an amount Lactobacillus rhamnosus.

3. The oral multi-component formulation of claim 2, wherein the Lactobacillus acidophilus is Lactobacillus acidophilus and the Lactobacillus rhamnosus is Lactobacillus rhamnosus.

4. The oral multi-component formulation of claim 2, wherein a ratio of the amount of Lactobacillus acidophilus to the amount of Lactobacillus rhamnosus is about 4:1.

5. The oral multi-component formulation of claim 1, wherein the at least one nutritional supplement ingredient comprises an amount folic acid or a pharmaceutically acceptable salt of folate.

6. The oral multi-component formulation of claim 2, wherein the at least one nutritional supplement ingredient comprises an amount of folic acid or a pharmaceutically acceptable salt of folate.

7. The oral multi-component formulation of claim 4, wherein the at least one nutritional supplement ingredient comprises an amount folic acid or a pharmaceutically acceptable salt of folate.

8. The oral multi-component formulation of claim 1, wherein the probiotic composition layer further comprises an amount of lactoferrin.

9. The oral multi-component formulation of claim 6, wherein the probiotic composition layer further comprises an amount of lactoferrin.

10. The oral multi-component formulation of claim 7, wherein the probiotic composition layer further comprises an amount of lactoferrin.

11. A method of balancing the vaginal microbiome, the method comprising:
identifying a subject with a pH level above about 4.5;
administering an oral multi-component formulation, wherein the oral multi-component formulation comprises:
a probiotic composition layer, wherein the probiotic composition layer comprises an amount of at least one Lactobacillus species;
a supplement composition layer, wherein the supplement composition layer comprises an amount of at least one nutritional supplement ingredient; and
a middle barrier layer disposed between the probiotic composition layer and the supplement composition layer, wherein the probiotic composition layer and the supplement composition layer are positioned on opposing sides of the middle barrier layer.

12. The method of claim 11, wherein the oral multi-component formulation is administered once per month.

13. The method of claim 12, wherein the oral multi-component formulation is administered once every day for 15 consecutive days.

14. The method of claim 11, wherein the probiotic composition layer comprises an amount of Lactobacillus acidophilus and an amount Lactobacillus rhamnosus, and the at least one nutritional supplement ingredient comprises an amount of folic acid or a pharmaceutically acceptable salt of folate.

15. The method of claim 11, wherein the probiotic composition layer further comprises an amount of lactoferrin.

16. A method of treating, preventing, or ameliorating at least one symptom selected from the group consisting of vaginal odor, vaginal discharge, vaginal itching, painful urination, and any combination thereof, the method comprising:
identifying the at least one symptom in the subject;
administering an oral multi-component formulation, wherein the oral multi-component formulation comprises:
a probiotic composition layer, wherein the probiotic composition layer comprises an amount of at least one Lactobacillus species;
a supplement composition layer, wherein the supplement composition layer comprises an amount of at least one nutritional supplement ingredient; and
a middle barrier layer disposed between the probiotic composition layer and the supplement composition layer, wherein the probiotic composition layer and the supplement composition layer are positioned on opposing sides of the middle barrier layer.

17. The method of claim 16, wherein the subject has bacterial vaginosis.

18. The method of claim 16, wherein the subject does not have bacterial vaginosis.

19. The method of claim 16, wherein the probiotic composition layer comprises an amount of Lactobacillus acidophilus and an amount Lactobacillus rhamnosus, and the at least one nutritional supplement ingredient comprises an amount of folic acid or a pharmaceutically acceptable salt of folate.

20. The method of claim 16, wherein the probiotic composition layer further comprises an amount of lactoferrin.

21. A method of regulating, restoring, or balancing the vaginal pH of a subject, the method comprising: identifying a subject in need thereof; administering an oral formulation to regulate, restore, or balance the vaginal pH of the subject.

22. The method of claim 21, wherein the vaginal pH of the subject is regulated, restored, or balanced between about 3.8 and 5.0.
